(19) United States
(12) Patent Application Publication
(10) Pub. No.: US 2014/0127288 A1
(43) Pub. Date: May 8, 2014

(54) LIPOSOME CONTAINING PYRROLOQUINOLINE QUINONE AND SUGAR

(71) Applicant: Kazuto Ikemoto, Niigata-shi (JP)

(72) Inventor: Kazuto Ikemoto, Niigata-shi (JP)

(73) Assignee: MITSUBISHI GAS CHEMICAL COMPANY, INC., Chiyoda-ku (JP)

(21) Appl. No.: 14/118,018

(22) PCT Filed: May 17, 2012
(86) PCT No.: PCT/JP2012/062704

§ 371 Date: Jan. 9, 2014

(30) Foreign Application Priority Data
May 17, 2011 (JP) 2011-110639

(51) Int. Cl. A61K 8/14; A23L 1/30; A61K 8/49

(52) U.S. Cl. CPC A61K 8/14; A61K 8/4926; A23L 1/30
USPC 424/450; 514/292; 435/404; 426/648

(57) ABSTRACT

An object of the present invention is to provide a composition that enables extension of the concentration range in which pyrroloquinoline quinone exerts cell growth-promoting function, and an efficient method of producing the composition. According to the present invention, there is provided a liposome composition comprising liposomes individually containing pyrroloquinoline quinone or a salt thereof and sugar, 50% or more of the liposomes having a particle diameter in the range of from 1 to 10 μm, and a method of producing the composition.

### CROSS-REFERENCE TO RELATED APPLICATION

This application enjoys the benefit of Japanese Patent Application No. 2011-110639, filed on May 17, 2011. The disclosure of this earlier application is incorporated herein by reference in its entirety.

### TECHNICAL FIELD

The present invention relates to technology for a liposome containing pyrroloquinoline quinone. Specifically, the present relates to a liposome composition, which enables extension of the concentration range of pyrroloquinoline quinone in which the function thereof is exhibited, and a method for producing the same.

### BACKGROUND ART

Pyrroloquinoline quinone (hereinafter sometimes referred to as “PQQ”) has been proposed as a possible new vitamin, and has attracted much attention as a useful material for food with enhanced nutritional function, dietary supplements, cosmetics, etc. Moreover, PQQ is present not only in bacteria but also in eukaryotic molds and yeasts and plays an important role as a cofactor. Also, PQQ has been found to have many physiological activities such as cell growth-promoting activity, anti-cataract activity, hepatic disease-preventing and therapeutic activity, wound healing activity, antiallergic activity, reverse transcriptase-inhibiting activity, glyoxalase I-inhibiting activity-anticancer activity, and the like.

PQQ can be obtained by subjecting PQQ obtained by methodologies such as organic chemical syntheses and fermentation processes to chromatography and concentrating the PQQ fraction in the effluent to crystallize PQQ by crystallization, followed by drying the crystallized PQQ.

A liposome generally means a capsule structure composed of lipid membranes consisting of a phospholipid, and an aqueous phase is encapsulated inside the capsule structure. A phospholipid molecule with a pine needle-like shape has two properties; the head part with hydrophilicity, and its leaf-like part with hydrophobicity. Therefore, when the molecule is released in water, its hydrophilic part is attracted to a water molecule to form a liposome. A liposome can encapsulate water-soluble components in its hydrophilic part and oil-soluble components in its hydrophobic part. Liposomes have been noted as a method for administration of drugs mainly in the field of medicine. In general, it is known that liposomes have the advantage of the improving the absorbability, dispersibility, and stability of drugs. Moreover, they have been widely used in oral administration and in applications to the skin.

To date, it has been known that the addition of PQQ to a culture of fibroblasts has an effect on DNA synthesis and cell proliferation. (Non-patent documents 1 and 2). This proliferation activity results from activation of cells, which means that this is a field expected to be applied to cosmetic products and food products. This PQQ activity is exhibited in a certain range of concentrations. However, reduction of the concentration and a wide variety of applications are demanded. The wide variety of usage of PQQ makes management easy, and facilitates the design of compositions in cosmetic products, food products, culture media, and the like. For example, the concentration of PQQ in which its usefulness is exhibited changes when different types of cells are used. Thus, the concentration of PQQ is required to be adjusted depending on its usage. Such operations, however, require a lot of efforts and such control of cancentrations is difficult in cosmetic products and the like. Moreover, its usage at high concentrations increases the risk of inhibiting cell proliferation; therefore, technologies to lower this type of risk have been required, particularly for liposomes with increased absorbability.

Liposomalization is generally used to increase the absorption of drugs. However, extending the concentration range of it is not known, nor is the effect on augmentation of cell proliferation specific to PQQ.

A fuel cell using PQQ as an electron mediator and an enzyme immobilized as a liposome has been heretofore developed (Patent document 1). Moreover, PQQ was proposed for use in a composition for the administration of an S-nitrosyl compound to mammals. However, there is no specific example and no description of a production method thereof (Patent document 2). Similarly, there have been documents which reported that PQQ can be administered in the form of a liposome, or added in the form of a liposome (Patent documents 3, 4, 5). However, there is no specific example. Therefore, these documents cannot provide any solution to the problem required to be solved in the present invention. Phospholipid has high viscosity as it is, so it is generally dissolved in a solvent such as chloroform to form a liquid film inside a flask, which is then dispersed using ultrasound to form a liposome. However, this method suffers from low productivity and there is a risk of remaining solvent. Thus, liposomes containing PQQ, a composition having improved functionality through such liposomes, and a method for producing them are needed. Moreover, PQQ having increased absorption capacity and functionality is still needed.

### PRIOR ART DOCUMENTS

### Patent Document

Patent document 1: Japanese Unexamined Patent Application Publication No. 2006-508519
Patent document 2: Japanese Unexamined Patent Application Publication No. 2001-518096
Patent document 3: Japanese Patent Application Laid-Open Publication No. 2009-221206
Patent document 4: Japanese Patent Application Laid-Open Publication No. 2006-335651
Patent document 5: Japanese Unexamined Patent Application Publication No. 2005-530786

### Non-Patent Document

Non-patent document 1: Life Science, Vol 52, p1902-1915 (1993)
Non-patent document 2: Int. J. Molecular Med., Vol 19, 765-770 (2007)

### SUMMARY OF INVENTION

### Problem to be Solved by the Invention

The present inventors have found that a liposome composition comprising liposomes individually containing pyrroloquinoline quinone and sugar, 45% or more of the liposomes having a particle diameter in the range of from 1 to 10 μm, which has been obtained by preparing a solution containing pyrroloquinoline quinone, sugar, and a lipid component at 40° C. or higher, exhibits cell growth-promoting function in a wide range of concentrations. The present invention is based on this finding.

An object of the present invention is to provide a liposome composition that enables extension of the concentration range of pyrroloquinoline quinone in which its function is exhibited, and an efficient method of production thereof.

### Means for Solving the Problem

According to the present invention, the following inventions are provided:

(1) A liposome composition comprising liposomes individually containing pyrroloquinoline quinone represented by the following formula (I),

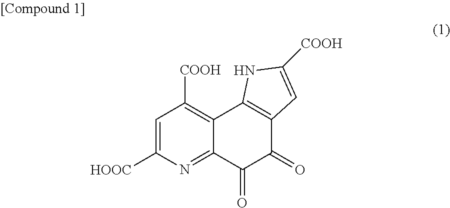 [SMILES: O=C(O)c1cc(C(=O)O)c2c(n1)C(=O)C(=O)c1cc(C(=O)O)[nH]c1-2]

or a salt thereof and sugar, 45% or more of the liposomes having a particle diameter in the range of from 1 to 10 μm.
(2) The liposome composition according to (1), wherein the volume mean particle diameter of the liposomes is in the range of 0.5 to 20 μm.
(3) The liposome composition according to (1), wherein the sugar is selected from the group consisting of monosaccharides, disaccharides, oligosaccharides, polysaccharides, and sugar alcohols.
(4) The liposome composition according to (1), wherein the sugar is sorbitol or xylitol.
(5) A food product comprising the liposome composition according to any of (1) to (4).
(6) A pharmaceutical product comprising the liposome composition according to any of (1) to (4).
(7) A culture medium comprising the liposome composition according to any of (1) to (4).
(8) A method for producing a liposome composition, comprising the step of preparing a solution comprising pyrroloquinoline quinone represented by the following formula (I) or a salt thereof, sugar, and a lipid component at 40 to 190° C.

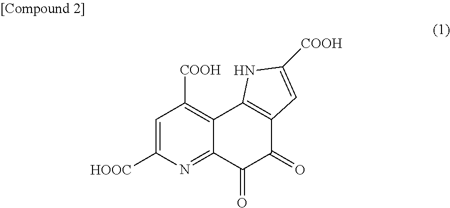 [SMILES: O=C(O)c1cc(C(=O)O)c2c(n1)C(=O)C(=O)c1cc(C(=O)O)[nH]c1-2]

(9) The method for production according to (8), wherein the pH of the solution is 8 or lower.
(10) The method for production according to (8), wherein the concentration of the solution of pyrroloquinoline quinone or a salt thereof is in the range of 0.0001 to 2% by weight, and the concentration of the sugar is in the range of 0.5 to 50% by weight.
(11) The method for production according to (8), wherein the weight ratio among pyrroloquinoline quinone or a salt thereof, sugar, the lipid component in a solution is in the range of 1:1 to 200:0.1 to 30.
(12) The method for production according to (8), further comprising the step of homogenizing the solution.
(13) A liposome composition prepared by the production method according to (8).

### Advantageous Effects of Invention

The present invention has the advantage of providing a liposome composition containing highly stable PQQ with a wide concentration range in which cell growth-promoting function is exhibited, and a method of producing the same.

### BRIEF DESCRIPTION OF DRAWINGS

FIG. 1 shows the particle size distribution of 2% sorbitol liposomes in Example 1.

FIG. 2 shows the particle size distribution of 10% sorbitol liposomes in Example 2.

FIG. 3 shows the particle size distribution of 20% sorbitol liposomes in Example 3.

FIG. 4 shows the particle size distribution of 40% sorbitol liposomes in Example 4.

FIG. 5 shows the particle size distribution of 0% sorbitol liposomes in Comparative Example 1.

FIG. 6 shows the particle size distribution of 10% sorbitol liposomes in Comparative Example 2.

FIG. 7 shows the particle size distribution of 10% sorbitol liposomes with small particle size in Comparative Example 3.

FIG. 8 shows a study of cell proliferation using cell in culture.

FIG. 9 shows the particle size distribution of 10% sorbitol liposomes in Example 5.

### DESCRIPTION OF EMBODIMENTS

The term “liposome” in this description refers to a closed vesicle composed of a lipid bilayer that encapsulates an aqueous phase inside.

The term “liposome composition” in this description refers to a composition comprising a plurality of liposomes. The liposome composition is preferably a liposomal dispersion liquid.

In the present invention, a liposome contains the free form of PQQ having a structure represented by the following formula (I) or a salt thereof, and sugar inside the membrane of the liposome.

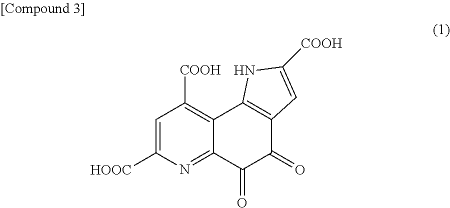 [SMILES: O=C(O)c1cc(C(=O)O)c2c(n1)C(=O)C(=O)c1cc(C(=O)O)[nH]c1-2]

Pyrroloquinoline quinone used in the present invention can be used as either pyrroloquinoline quinone (the free form) or a salt of pyrroloquinoline quinone.

“A salt of pyrroloquinoline quinone” used in the present invention includes an alkali metal salt, an alkali earth metal salt, and an ammonium salt of pyrroloquinoline quinone, preferably an alkali metal salt.

An alkali metal salt of pyrroloquinoline quinone used in the present invention includes a sodium salt, a potassium salt, a lithium salt, a cesium salt, a rubidium salt, and the like. Regarding availability, a sodium salt and a potassium salt are more preferable. Pyrroloquinoline quinone may be an alkali metal salt that is substituted with 1 to 3 alkali metals. It can be used as a mono-alkali metal salt, a di-alkali metal salt, or a tri-alkali metal salt, preferably a di-alkali metal salt. As an alkali metal salt of pyrroloquinoline quinone, a disodium salt and a dipotassium salt are in particular preferred.

As pyrroloquinoline quinone or a salt thereof used in the present invention, the free form, a disodium form, and a dipotassium form are in particular easily available and easy to use.

Pyrroloquinoline quinone or a salt thereof used in the present invention can be commercially available or produced by a publicly known method.

Sugar is preferably water-soluble. A monosaccharide, a disaccharide, an oligosaccharide, a polysaccharide, and a sugar alcohol can be used. Specifically, examples of the monosaccharide include glyceraldehyde, threose, arabinose, xylose, ribose, ribulose, xylulose, glucose, mannose, galactose, tagatose, allose, altrose, gulose, idose, talose, sorbose, psicose, fructose, and the like. As for the disaccharide, examples include trehalose, sucrose, lactose, and the like. As for the oligosaccharide, examples include maltotriose, raffinose, cyclodextrin, and the like. As for the polysaccharide, examples include starch syrup, hydrogenated starch syrup, and the like. As the sugar alcohol, examples include threitol, erythritol, adonitol, arabitol, xylitol, talitol, sorbitol, mannitol, iditol, dulcitol, inositol, and the like. Monosaccharide, disaccharide, and sugar alcohol are preferable, more preferably sugar alcohol. The monosaccharide is preferably glucose. The disaccharide is preferably sucrose. The sugar alcohol is preferably sorbitol and xylitol. Sugar alcohol is prepared by hydrogenation of common sugars and starch syrup, and does not have an active carbonyl group. Therefore, they are heat- and acid-stable, and have low calories. Addition of the sugar can expand the concentration range of PQQ in which its functiona is exhibited.

The sugar used in the present invention can be commercially available or can be produced by a publicly known method.

A liposome (a liposome membrane) is composed of a lipid component, for example, a phospholipid or glycolipid alone or in a mixture thereof.

As the phospholipid, there is phosphatidylcholine as a dominant component of the phospholipid present in living organisms, and is also referred to as lecithin. As the phospholipid, egg-yolk lecithin, soybean lecithin, purified soybean lecithin, phosphatidylcholine, phosphatidylethanolamine, phosphatidylserine, sphingomyelin, dicetyl phosphate, stearylamine, phosphatidyl glycerol, phosphatidic acid, phosphatidyl inositol amine, cardiolipin, ceramide phosphorylethanolamine, ceramide phosphorylglycerol and a mixture thereof can be used. It is preferable to use a purified phospholipid as the phospholipid.

In the present invention, commercially available phosphatidylcholine can be used as the phospholipid. For example, COATSOME NC-21 from NOF Corporation (hydrogenated soybean phospholipid, PC content is 90% or more), NIKKOL Lecinol S-10EX from Nikko Chemicals Co., LTD. (hydrogenated soybean phospholipid, PC content is 95% or more) can be used. Egg-yolk lecithin, soybean lecithin, purified soybean lecithin, and hydrogenated soybean phospholipids are also readily available, so they are preferable.

As the glycolipid, digalactosyl diglyceride, galactosyl diglyceride sulfate, galactosyl ceramide, galactosyl ceramide sulfate, lactosyl ceramide, ganglioside G7, ganglioside G6, ganglioside G4, digalactosyl ceramide, and a mixture thereof can be used.

As the membrane constituent of the liposome, a sterol can be added with lipids such as phospholipid and glycolipid. The upper limit of the added sterol with respect to the phospholipid or glycolipid is preferably ⅕ by weight; and 1/10 by weight is more preferable. Cholesterol is the most preferred as the sterol, but other sterols can be used.

Liposomes can be produced by a conventional method. For example, lecithin is dissolved in an organic solvent, for example chloroform, and subsequently the solvent is evaporated using a rotary evaporator to form a lipid film on the flask wall, followed by the addition of a PQQ solution to prepare liposomes. However, this procedure is complicated, and there is a risk of leaving a toxic organic solvent. This process also requires an analysis of the toxic organic solvent and is hence costly; therefore, it is not the most suitable method.

The liposome composition of the present invention can be produced by preparing a solution containing pyrroloquinoline quinone or a salt thereof, sugar, and a lipid component at a temperature in the range of 40 to 190° C.

The solution can be obtained by mixing pyrroloquinoline quinone or a salt thereof, sugar, a lipid component, and a solvent. Typically, the process can be conducted by adding pyrroloquinoline quinone or a salt thereof, sugar and a lipid component to the solvent. The order of the addition is not particularly limited.

The solvent used herein is not particularly limited so long as the reaction progresses, and water, ethanol and the like can be used. Water (an aqueous solution) is preferable since there is less concern if it remains in a product.

The concentration of pyrroloquinoline quinone or a salt thereof in the solution can be, for example, in the range of 0.0001 to 2% by weight, but preferably in the range of 0.01 to 1.5% by weight, and more preferably in the range of 0.1 to 1% by weight.

The concentration of the sugar in the solution can be, for example, in the range of 0.5 to 50% by weight, however, preferably in the range of 2 to 50% by weight, more preferably in the range of 5 to 30% by weight, and further preferably 10 to 20% by weight.

The weight ratio of pyrroloquinoline quinone or a salt thereof to the sugar in the solution can be in the range of 1:0.1 to 200, preferably in the range of 1:1 to 200, more preferably in the range of 1:1 to 100, further preferably in the range of 1:10 to 100, and in particular preferably in the range of 1:30 to 70.

The concentration of the lipid component in the solution can be in the range of 0.001 to 10% by weight. For example, when phospholipid is used as the lipid component, it can be in the range of 0.001 to 10% by weight. It is, however, preferably in the range of 0.01 to 8% by weight, and more preferably in the range of 0.1 to 5% by weight.

The weight ratio of pyrroloquinoline quinone or a salt thereof to the lipid component can be in the range of 1:0.1 to 30, and preferably in the range of 1:1 to 20.

The weight ratio among pyrroloquinoline quinone or a salt thereof, the sugar, the lipid component can be in the range of 1:0.1 to 200:0.1 to 30, preferably in the range of 1:1 to 200:0.1 to 30, more preferably in the range of 1:1 to 100:1 to 20, further preferably in the range of 1:10 to 100:1 to 20, and most preferably in the range of 1:30 to 70:1 to 20. In particular, the weight ratio among pyrroloquinoline quinone or a salt thereof, the sugar, the phospholipid in the solution can be in the range of 1:0.1 to 200:0.1 to 30, preferably in the range of 1:1 to 200:0.1 to 30, more preferably in the range of 1:1 to 100:1 to 20, further preferably in the range of 1:10 to 100:1 to 20, and in particular preferably in the range of 1:30 to 70:1 to 20.

The pH of the solution obtained can be adjusted to 8 or lower, but preferably 1 to 6, more preferably 2 to 5, and further preferably 3 to 4. Acidic substances (e.g., hydrochloric acid, acetic acid, etc.) and alkaline substances (e.g., sodium hydroxide, sodium bicarbonate, etc.) can be used to adjust pH.

The temperature of the solution obtained can be in the range of room temperature to 190° C., preferably in the range of 40 to 190° C., more preferably in the range of 60 to 190° C., further preferably in the range of 60 to 150° C., furthermore preferably in the range of 60 to 120° C., in particular preferably in the range of 60 to 100° C., and in particular more preferably in the range of 60 to 80° C. The temperature of the solution can be adjusted by changing the solvent temperature.

The solution obtained can be subjected to a step of adjusting the temperature. This “adjusting” of temperature can be conducted by warming (including holding of the temperature), leaving as it is, or cooling in accordance with the temperature of the solution obtained. Specifically, the temperature of the solution obtained can be adjusted in the range of 40 to 190° C., preferably in the range of 60 to 190° C., more preferably in the range of 60 to 150° C., further preferably in the range of 60 to 120° C., furthermore preferably in the range of 60 to 100° C., and in particular preferably in the range of 60 to 80° C.

The temperature of the solution obtained in advance in the range of 60 to 190° C. (more preferably in the range of 60 to 150° C., further preferably in the range of 60 to 120° C., furthermore preferably in the range of 60 to 100° C., and in particular preferably in the range of 60 to 80° C.), is preferably maintained at a temperature of 60° C. or higher (in the range of 60 to 190° C., preferably in the range of 60 to 150° C., more preferably in the range of 60 to 120° C., further preferably in the range of 60 to 100° C., and in particular preferably in the range of 60 to 80° C.).

The solution having an adjusted temperature can be further subjected to a step of homogenization. The step of homogenization means a step of highly dispersing the components in the solution obtained.

In the step of homogenization, the solution can be maintained at the adjusted temperature, for example, in the range of 40 to 190° C., preferably in the range of 60 to 190° C., more preferably in the range of 60 to 150° C., further preferably in the range of 60 to 120° C., furthermore preferably 60 to 100° C., and in particular preferably 60 to 80° C.

In the step of homogenization, a homogenizer (emulsifier) can be used. Among emulsifiers, a stirring emulsifier such as NISSEI AM-3 homogenizer from Nippon Seiki Co., Ltd., Ultra-Turrax T 25 from IKA, and the like can be used.

In the step of homogenization, a high-pressure emulsifier can be used. As the high-pressure emulsifier, a thin-film spin system high-speed homo mixer (T.K Filmix) from PRIMIX Corporation, an ultrahigh-pressure homogenizer (microfluidizer) from Microfluidics, Inc., a mixer utilizing internal shearing force (Clearmix) from M Technique Co., Inc., a wet-type, media-less pulverizer (Nanomizer) from Yoshida Kikai Co., LTD., and the like can be used.

The conditions of the homogenization can be optionally decided based on the machine used. For example, when NISSEI AM-3 homogenizer from Nippon Seiki Co., Ltd. is used, it can be spun for 0.5 to 180 minutes (preferably 1 to 60 minutes) at a speed of 1000 to 10000 rpm.

The step of homogenization can be further conducted at room temperature. The procedure and conditions for homogenization are as mentioned above, however, the duration of the homogenization step can be preferably in 10 minutes or less.

Preferably, liposomes can be produced by adding a sterol, a polyhydric alcohol, and a pH adjuster to an aqueous solution containing PQQ in an amount of 0.0001 to 2% by weight, and sugar in an amount of 2 to 50% by weight as needed, and warming the resultant solution at 60 to 190° C., followed by dispersing the additives using a homogenizer. Use of a homogenizer may enable production of liposomes in high productivity. Herein, the upper limit of the concentration of PPQ is the same as the solubility of PQQ. When the concentration is higher than this limit, PQQ easily precipitates. When the PQQ concentration is lower than the lower limit, the PPQ function cannot be expected.

Because PQQ is not stable under alkaline conditions, the pH is preferably 8 or lower, and more preferably 1 to 6. If the pH is 8 or higher, PQQ will be decomposed. The stability can be attained by making the solution acidic, but it is difficult to obtain a solution containing PQQ at high concentration since the solubility of PQQ decreases under acidic conditions.

The liposome composition of the present invention can be obtained through these steps.

The particle diameter of liposomes in the present invention is in the range of 0.5 to 100 μm as is used usually. More preferably, the particle diameter of liposomes is in the range of 1 to 10 μm.

In the present invention, liposomes having a particle diameter in the range of 1 to 10 μm are preferably in an amount of 45% or more of the total liposomes, more preferably 50% or more, further preferably in the range of 80 to 100%, and in particular preferably in the range of 90 to 100%. Herein “%” means “volume %”. Small size liposomes (e.g., liposomes having a particle diameter ranging from 0.1 to 0.9 μm) are effective in increasing absorbability, but ineffective in extending the concentration range in which the function of pyrroloquinoline quinone is exhibited. Large size liposomes (e.g., liposomes having a particle diameter ranging from 20 to 200 μm) are not preferable due to their low absorbability and difficulty in the production.

In the present invention, the volumetric average particle diameter of liposomes can be in the range of 0.5 to 20 μm, and preferably 1 to 10 μm.

In the present invention, the particle diameter can be measured by a publicly known instrument. For example, a particle size distribution measuring instrument (e.g., SEISHIN LMS-350, Seishin Enterprise Co., Ltd.) can be used.

Meanwhile, the particle diameter of liposomes in the present invention was measured in the form of dispersion in water as mentioned in Examples.

The particle diameter of the liposomes can be controlled by selecting raw materials, conditions of production, and the like as described above. Alternatively, liposomes once prepared can be easily controlled by filtration. The purification and size control of liposomes can be conducted by treatments such as dialysis, freezing and thawing, lyophilization, centrifuge separation, and the like.

It is well known that small size liposomes have excellent absorbability. However, even large size liposomes have excellent absorbability in accordance with the present invention. Additionally, the present invention enables extension of the optimum concentration range in which the distinctive function of PQQ is exhibited.

In the present invention, the term “function” of pyrroloquinoline quinone refers to a cell growth-promoting function and antioxidative property; in particular it means a cell growth-promoting function.

Liposomes of the present invention may be in the form of a liposome composition in which liposomes coexist with the free form of PQQ or a salt thereof and sugar outside the liposomes. In the process of preparing the liposome composition, a sterol, a polyoxyethylene sterol ether, a polyhydric alcohol, a pH adjuster, a nonionic surfactant, an anionic surfactant, a cationic surfactant, an amphoteric surfactant, an oil, a moisturizing agent, a water-soluble polymer, an antioxidant, an ultraviolet absorbing agent, a chelating agent, an antiseptic agent, an antibacterial agent, a colorant, a fragrance, and the like can be combined within a range in which the effect of the present invention is not adversely affected. Furthermore, vitamins such as coenzyme Q10, ascorbic acid derivatives, tocopherol, arachidonic acid, DHA, retinol derivatives and the like; and plant extracts such as ginkgo leaf extract and Cistanche tubulosa extract may be combined.

The liposome composition of the present invention may be in the form of any one of an aqueous solution, an oil-in-water emulsified composition, a water-in-oil emulsified composition, a multi-emulsified composition, a multi-lamellar agent and the like. Herein, the aqueous solution means an aqueous solution in which liposomes are dispersed.

Furthermore, other pharmaceutically acceptable materials for drug formulation may be added and mixed according to a conventional method. The materials for drug formulation that can be added are not in particular limited. For example, an emulsifying agent, a tensing agent, a buffer agent, a solubilizing agent, an odor improving agent, an antiseptic agent, a stabilizing agent, and an antioxidant can be cited.

The preservation method for the liposome composition of the present invention is not in particular limited. For example, techniques such as preservation at low temperature, anaerobic preservation in an airtight container, light shield storage, and the like can be applied. The composition of the present invention prepared in these manners can be stably preserved without precipitation when it is stored in a refrigerator or at room temperature.

Liposomes of the present invention can be used for wide variety of applications in medical treatment, cosmetic products, food products, horticulture, dairy products, and the like. Specific embodiments include injections, infusion solutions, liquid formulations, ophthalmic solutions, liquid medicines for oral administration, lotions, hair tonics, cosmetic emulsions, sprays, aerosols, liquid medicines, liquid fertilizers, solutions for preservation, and the like.

Animal cell culture is used for research and manufacturing of pharmaceutical drugs. The addition of the liposome composition of the present invention makes the manufacture and experiments of antibody drugs more efficient.

According to a preferred embodiment of the present invention, there is provided a liposome composition comprising liposomes containing pyrroloquinoline quinone or a salt thereof and sugar selected from the group consisting of monosaccharides, disaccharides, and sugar alcohols, 50% or more of the liposomes having a particle diameter in the range of from 1 to 10 μm.

According to a more preferred embodiment of the present invention, there is provided a liposome composition comprising liposomes containing pyrroloquinoline quinone or a salt thereof, and sugar selected from the group consisting of monosaccharides, disaccharides, and sugar alcohols, 90% or more of the liposomes having a particle diameter in the range of 1 to 10 μm.

According to a preferred embodiment of the present invention, there is provided a method of producing a liposome composition comprising the steps of: preparing a solution comprising pyrroloquinoline quinone or a salt thereof, sugar selected from the group consisting of monosaccharides, disaccharides, and sugar alcohols, and a phospholipid at a temperature in the range of 60 to 120° C. (preferably 60 to 80° C.) at a pH of 8 or lower; and homogenizing the solution.

According to a further preferred embodiment of the present invention, there is provided a method of producing a liposome composition comprising the steps of: preparing a solution comprising pyrroloquinoline quinone or a salt thereof, sugar selected from the group consisting of monosaccharides, disaccharides, and sugar alcohols, and a phospholipid in the weight ratio among 1:1 to 200:0.1 to 30 at a temperature in the range of 60 to 120° C. (preferably 60 to 80° C.) at a pH of 8 or lower; and homogenizing the solution.

According to the present invention, the following inventions are also provided.

[1] Liposomes containing pyrroloquinoline quinone represented by the following formula (I);

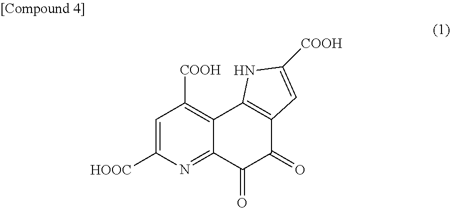 [SMILES: O=C(O)c1cc(C(=O)O)c2c(n1)C(=O)C(=O)c1cc(C(=O)O)[nH]c1-2]

or a salt thereof, and sugar, wherein the amount of the liposomes having a particle diameter in the range of 1 to 10 μm is 50% or more.
[2] The liposomes according to [1], wherein the sugar is sorbitol or xylitol.
[3] A food product containing the liposomes according to [2].
[4] Åpharmaceutical product containing the liposomes according to [2].
[5] A culture medium containing the liposomes according to [2].
[6] A method of producing liposomes comprising the steps of: preparing an aqueous solution containing pyrroloquinoline quinone and sugar dissolved therein at a pH of 8 or lower; and warming the solution at 60° C. to 190° C.
[7] The production method according to [6], wherein the concentration of pyrroloquinoline quinone or a salt thereof in the aqueous solution is in the range of 0.0001 to 2% by weight and that of the sugar is in the range of 2 to 50% by weight.
[8] The production method according to [7], further comprising the step of using a homogenizer.

### EXAMPLES

The present invention hereinafter is more in detail explained by Examples and Examples of preparation, however, the present invention is not limited to those.

### Reagents

Pyrroloquinoline quinone disodium from Mitsubishi Gas Chemical Company, Inc., COATSOME NC-21 (hydrogenated soybean phospholipid) from NOF Corporation, and other reagents from WAKO Pure Chemical Industries, Ltd. were used.

### Measurement of Particle Diameter of Liposomes

Liposomes were dispersed in water and the particle size distribution was measured by using a SEISHIN LMS-350 (Seishin Enterprise Co., Ltd.). The minimum detection limit on this instrument is 0.1 μm.

### Comparative Example 1

Preparation of a liposome composition

PQQ disodium (0.3 g) and COATSOME NC-21 (hydrogenated soybean phospholipid) (3.0 g) were mixed with water so that the total amount was adjusted to 100 g. In this case, the pH of the solution was 3.5. The solution obtained was treated by a NISSEI AM-3 homogenizer (Nippon Seiki CO., Ltd.) at a speed of 7000 rpm for 30 minutes while the solution was being warmed at 60° C. and higher, and then the temperature of the solution was reduced to room temperature followed by treatment at a speed of 7000 rpm for 10 minutes. After the treatment, water was added to compensate the amount of the water lost by evaporation. The resultant composition was referred to as a liposome composition of Comparative Example 1.

### Example 1-4

PQQ disodium (0.3 g) and COATSOME NC-21 (hydrogenated soybean phospholipid) (3.0 g) were used and sorbitol was added in an amount of 2, 10, 20 or 40 g, and the total amount was adjusted to 100 g with water. In this case, the pH of the solution was 3.5, and the temperature of the solution was 60° C. The solution obtained was treated to prepare a liposome composition of Examples 1 to 4 in the same manner as Comparative Example 1.

### Comparative Example 2

Preparation of a liposome composition containing sugar without PQQ

COATSOME NC-21 (hydrogenated soybean phospholipid) (3.0 g) and sorbitol (10 g) were mixed with water so that the total amount was adjusted to 100 g. A liposome composition was prepared by the same treatment as in Comparative Example 1.

### Comparative Example 3

Preparation of a liposome composition containing small sized particles

Soybean lecithin (0.3 g), PQQ disodium (0.3 g) and sorbitol (10 g) were mixed with water so that the total amount was adjusted to 100 g. A liposome composition was prepared by the same procedure as in Comparative Example 1.

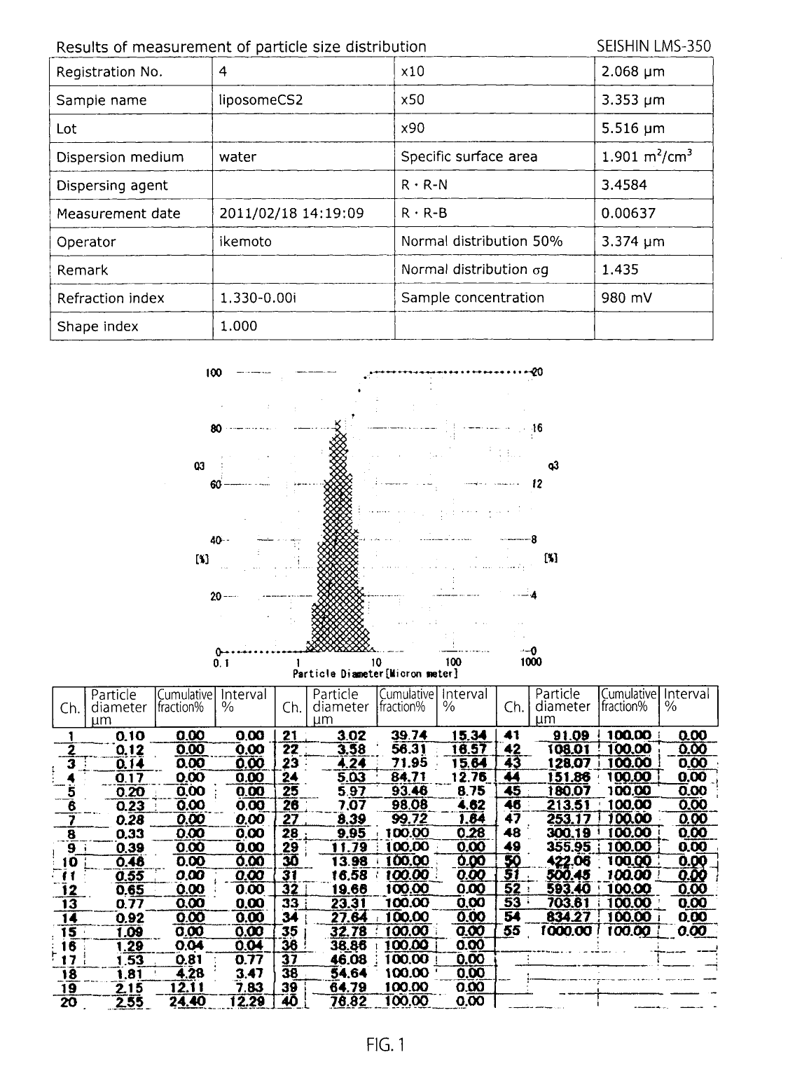

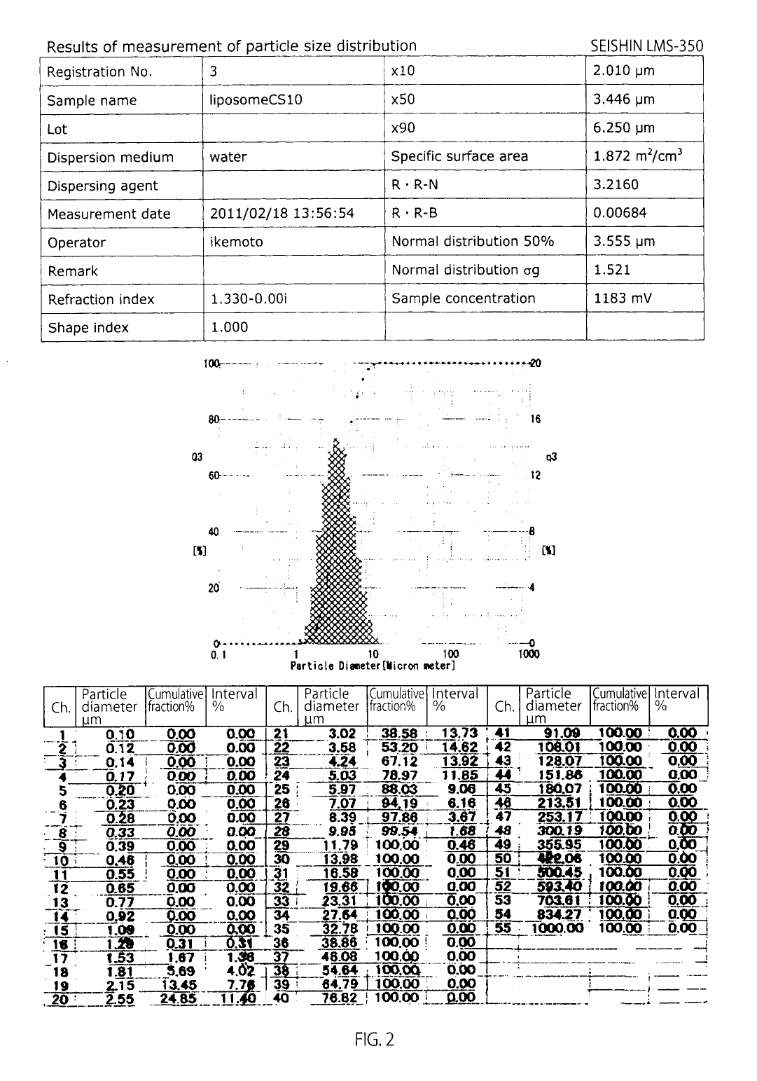

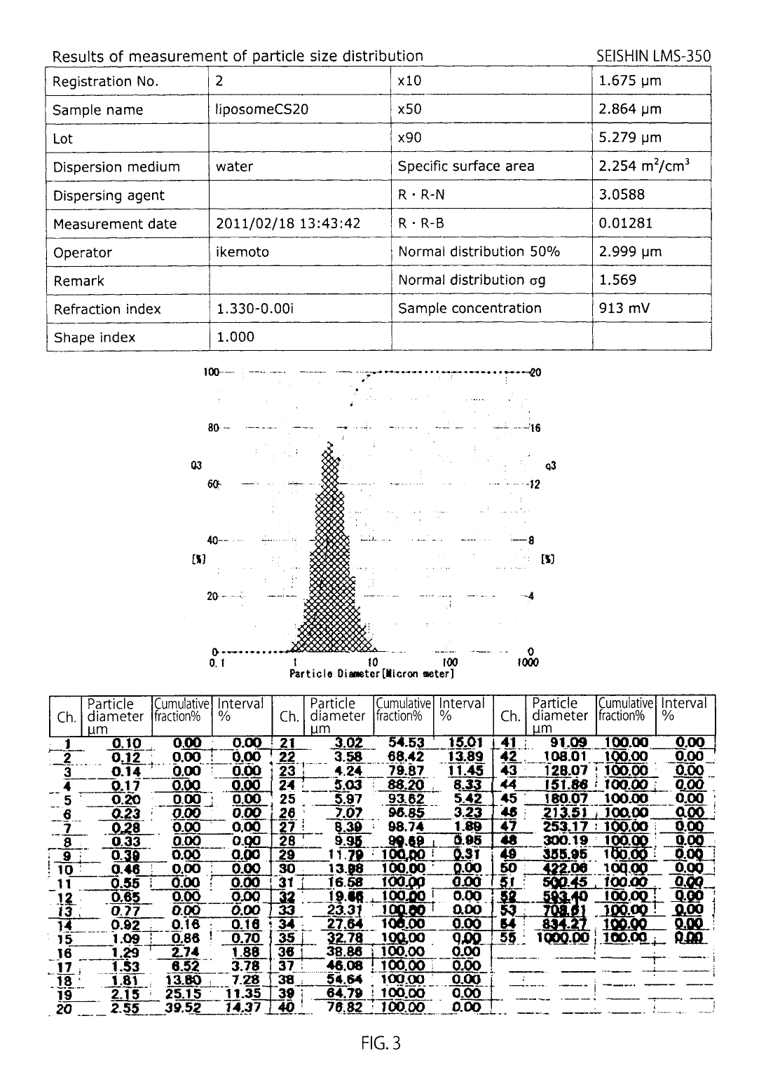

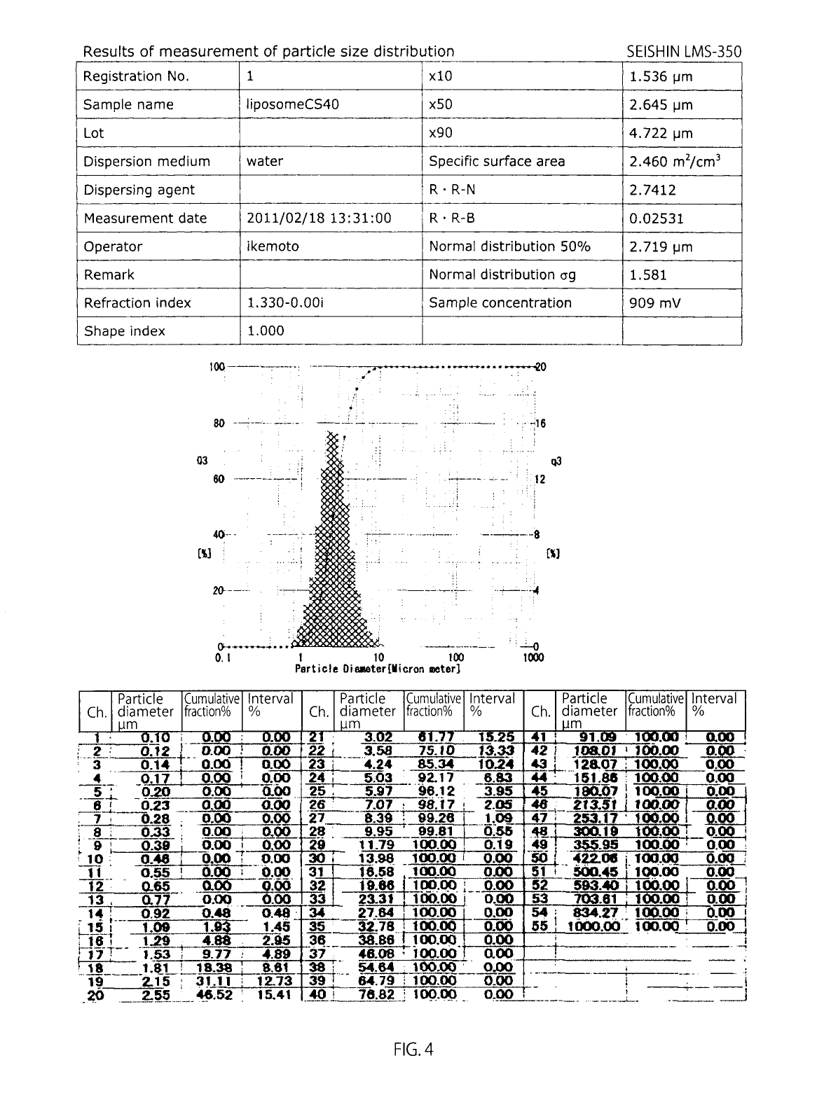

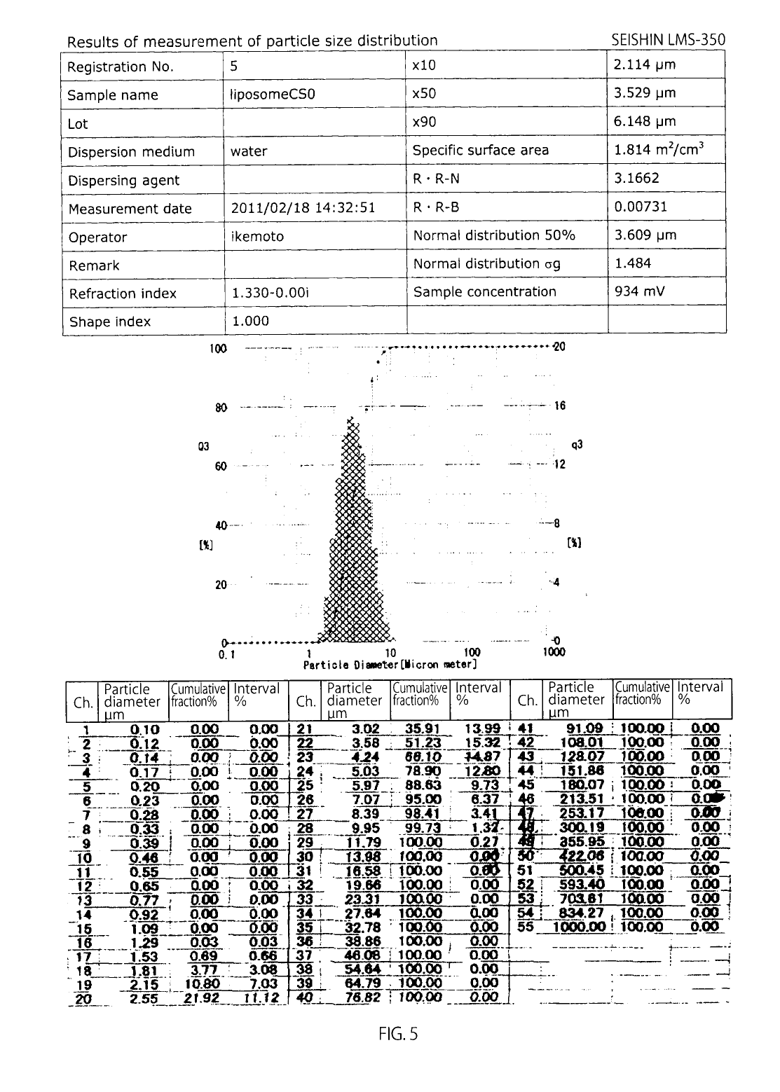

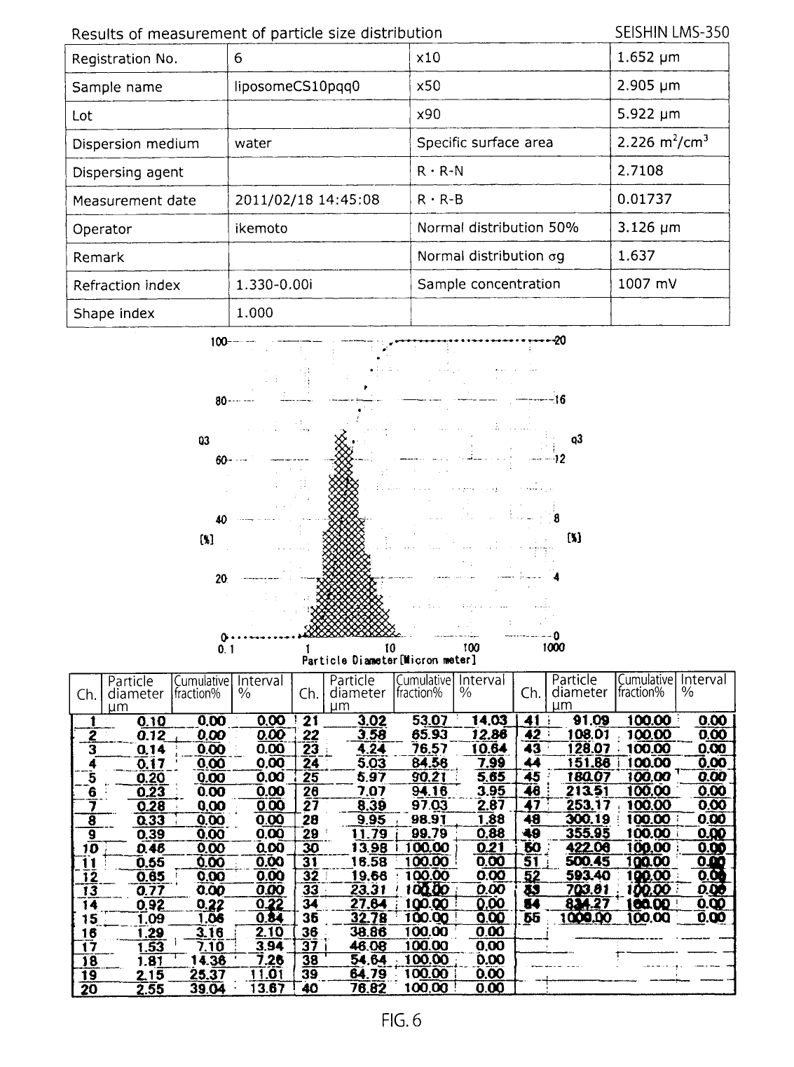

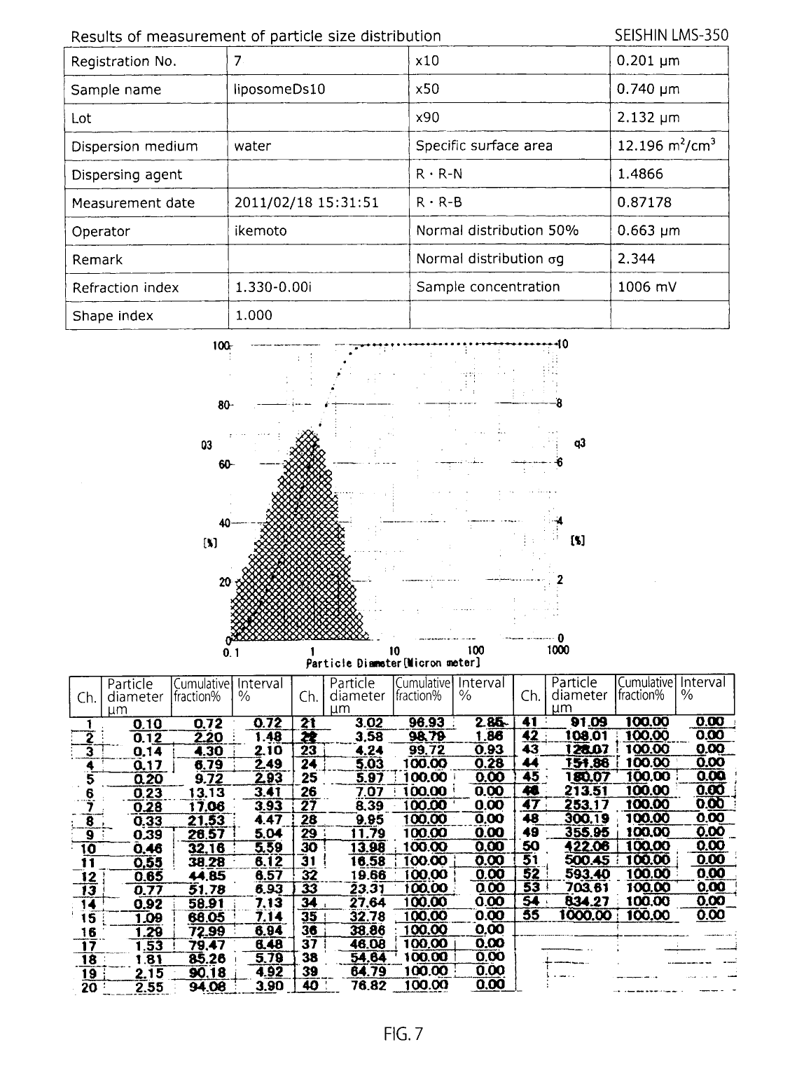

The results of measurement of the particle diameter of the liposomes prepared in Examples 1 to 4 and Comparative Examples 1 to 3 are shown in the following Table 1, and the results of each particle size distribution are shown in FIGS. 1 to 7.

TABLE 1
Results of measurement of particle diameters

| | | Particle | | Proportion |
|---|---|---|---|---|
| | | diameter- | Size- | of particles |
| | | Normal | Normal | between |
| | Sorbitol | distribution- | distribution- | 1-10 μm |
| | (%) | 50% (μm) | σg | (%) |
| Example 1 | 2 | 3.374 | 1.435 | 100 |
| Example 2 | 10 | 3.555 | 1.521 | 99.5 |
| Example 3 | 20 | 2.999 | 1.569 | 99.5 |
| Example 4 | 40 | 2.719 | 1.581 | 99.3 |
| Comparative | 0 | 3.609 | 1.484 | 99.7 |
| Example 1 | | | | |
| Comparative | 10 | 3.126 | 1.637 | 98.7 |
| Example 2 | | | | |
| Comparative | 10 | 0.663 | 2.344 | 41 |
| Example 3 | | | | |

From the results shown in FIGS. 1 to 6, it was clear that most liposomes in Examples 1 to 4 and Comparative Examples 1 to 2 had a particle diameter in the range of 1 to 10 μm.

The change of particle diameter by the addition of sorbitol was small, so it was demonstrated that the addition of sugar had little effect on the particle diameter of liposomes. It became also clear that the presence or absence of PQQ had little effect on the particle diameter.

In Comparative Example 3, liposomes containing small sized particles can be produced by using common soybean lecithin, and the effect of using small sized particles can be observed.

### Proliferation Study

Chinese hamster ovary cells (CHO-DHFR; obtained from Dainippon Sumitomo Pharma Co., Ltd.) was cultured in the culture medium, α-MEM+10% fetal calf serum, in an atmosphere of 5% CO₂ at 37° C.

A 96-well microplate (IWAKI & Co., Ltd.) was used and cells were added to each well at 5000 cells with 100 μl of the culture medium, and the microplate was incubated overnight. After removing the culture medium, a new culture medium containing the liposome composition prepared in Examples 1 to 4 and Comparative Examples 1 to 3 at a predetermined concentration was added to each well. After one day incubation, a test solution in a WST Assay Kit was reacted for 1 hour according to a procedure of the Kit (Dojindo Laboratories), and the absorbance of each well was measured at 450 nm. In WST Assay, the absorbance obtained is proportional to the number of live cells.

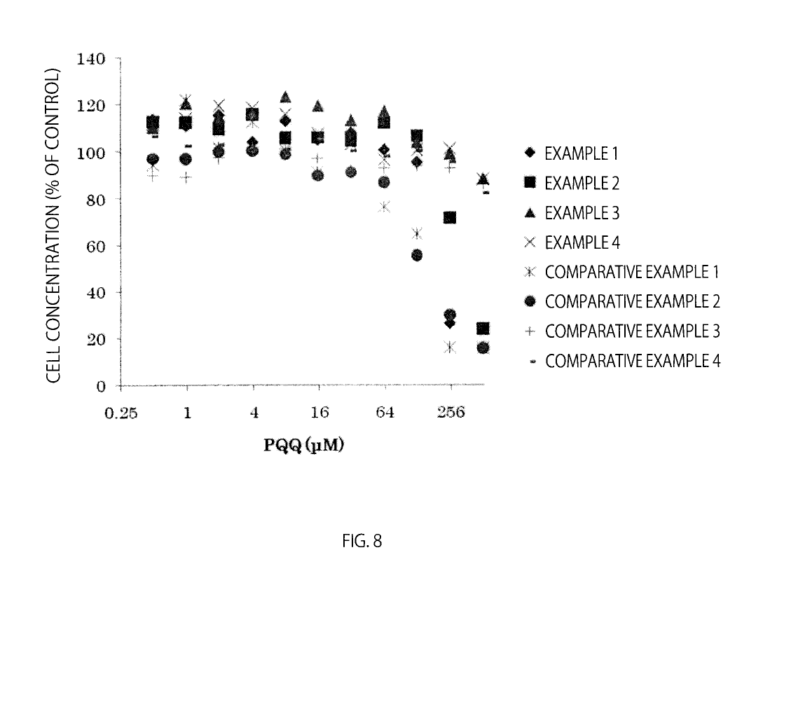

The liposome compositions prepared in Examples 1 to 4 and Comparative Examples 1 to 3 and PPQ disodium (for Comparative Example 4) were used as samples, and the samples were diluted by the culture medium and subjected to a test. The concentrations used in the test were 500, 250, 125, 62, 31, 16, 8, 4, 2, 1, 0.5, and 0 μM. In Comparative Example 2, the concentration of the liposome composition was used as the test concentration of the sample. The test was conducted for each sample twice and the average was calculated. The results are shown in FIG. 8. Values on the vertical axis are scaled to the number of cells obtained by incubating with the culture medium alone referred to as 100.

The additional concentration at which the total number of cells decreases approximately by 10% compared to control cells treated with culture medium alone is regarded as the concentration of growth inhibition (when this concentration decreases, the absorbability increases). Moreover, the additional concentration at which the total number of cells increases approximately 5% compared to control cells treated with the culture medium alone is regarded as the concentration of growth-promoting. The test results of each sample are shown in Table 2.

TABLE 2
Results of proliferation study

| | | Concentration | Growth- | |
|---|---|---|---|---|
| | | of growth | promoting | |
| | Sorbitol | inhibition | concentration | |
| | (%) | (μm) | (μm) | |
| Example 1 | 2 | 125 | 0.5 to 62 | |
| Example 2 | 10 | 125 | 0.5 to 125 | |
| Example 3 | 20 | 500 | 0.5 to 125 | |
| Example 4 | 40 | 500 | 0.5 to 62 | |
| Comparative | 0 | 62 | 1 to 31 | |
| Example 1 | | | | |
| Comparative | 10 | 500 | — | Without |
| Example 2 | | | | PQQ |
| Comparative | 10 | 31 | — | |
| Example 3 | | | | |
| Comparative | — | 500 | 16 | With PQQ |
| Example 4 | | | | |

The additional amount at which the total cell number decreases by 10% compared to the total number of cells treated with culture medium alone is 500 μM for PQQ disodium in Comparative Example 4, and 62 μM for the liposome composition without sorbitol in Comparative Example 1. In contrast to these results, the concentration of growth inhibition was 31 μM for the small sized liposome composition in Comparative Example 3. Because the absorbability was increased by liposomalization, the growth inhibition was observed at a low concentration.

Regarding the growth-promoting concentration, the liposome compositions in Examples 1 to 4 were effective at a wider range of concentration compared to that in Comparative Example 1.

When PQQ and sugar were added to the liposomes, the growth-promoting concentration increased and the growth inhibition effect decreased.

The liposome composition without the addition of PQQ in the presence of sugar in Comparative Example 2 revealed the effect on cell proliferation at a concentration equivalent to 500 μM. However, there was no effect at other concentrations. Moreover, in the small sized liposome composition in Comparative Example 3, the growth inhibition appeared at a low concentration, and no cell growth-promoting effect was observed (the concentration at which the cell growth-promoting effect is exhibited is likely lower than the tested concentration range).

Further, the range of concentration in which about 5% increase in the growth promotion due to PQQ was observed was unexpectedly extended by the increase in the addition of the sugar. The effect was observed from low concentrations in a wide range.

Extension of the concentration range in which the cell growth-promotion effect is exhibited is thought to facilitate a wide selection of the compositions in the case of the addition to culture media.

### Example 5

PQQ disodium (0.3 g), sorbitol (10 g) and COATSOME NC-21 (hydrogenated soybean phospholipid) (3.0 g) were mixed with water so that the total amount was adjusted to 100 g. In this case, the pH of the solution was 3.5, and the temperature was 40° C. While warming the resultant solution and eventually keeping the temperature at 60° C. or higher, the solution obtained was treated by a NISSEI AM-3 homogenizer at a speed of 7000 rpm for 30 minutes, and then the temperature was reduced to room temperature followed by treatment at a speed of 7000 rpm for 30 minutes. After the treatment, water was added to compensate the water lost by evaporation to adjust the total weight to 100 g.

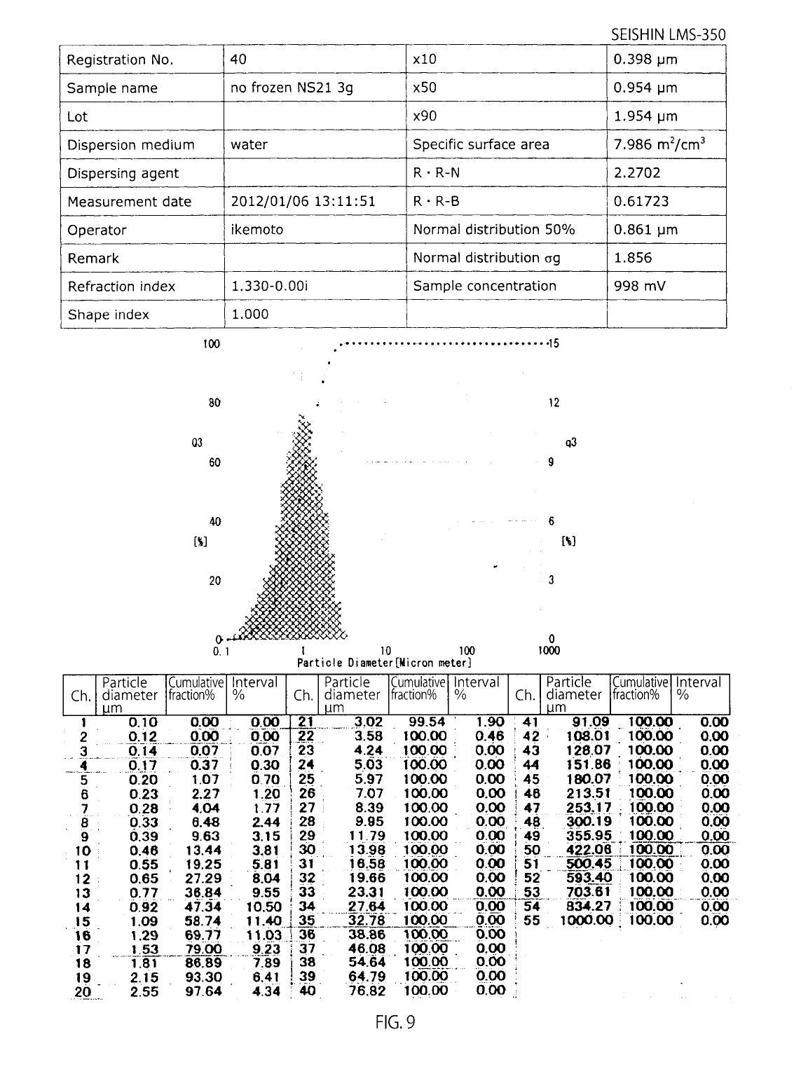

The measurement results of the particle diameter of the liposomes prepared in Example 5 are shown in the following Table 3, and the particle size distribution is shown in FIG. 9.

TABLE 3

| | | Particle | | Proportion |
|---|---|---|---|---|
| | | diameter- | Size- | of particles |
| | | Normal | Normal | between |
| | Sorbitol | distribution- | distribution- | 1-10 μm |
| | (%) | 50% (μm) | σg | (%) |
| Example 5 | 10 | 0.861 | 1.856 | 53 |

### Examples 6 and 7

PQQ disodium (0.3 g), sorbitol (50 or 0.5 g), and COATSOME NC-21 (hydrogenated soybean phospholipid) (3.0 g) were mixed with water so that the total amount was adjusted to 100 g. In this case, the pH of the solution was 3.5, and the temperature was 60° C. A liposome composition was prepared in the same way as in Examples 1 to 4.

The results of measurement of the particle diameter of the liposomes prepared in Examples 6 and 7 are shown in the following Table 4.

TABLE 4

| | | Particle | | Proportion |
|---|---|---|---|---|
| | | diameter- | Size- | of particles |
| | | Normal | Normal | between |
| | Sorbitol | distribution- | distribution- | 1-10 μm |
| | (%) | 50% (μm) | σg | (%) |
| Example 6 | 50 | 2.920 | 1.628 | 99.1 |
| Example 7 | 0.5 | 4.609 | 1.607 | 93.9 |

The results of the proliferation tests for Examples 5 to 7 are shown in the following Table 5.

TABLE 5

| | Growth-promoting |
|---|---|
| | concentration |
| | (μm) |
| Example 5 | 0.5-125 |
| Example 6 | 0.5-62 |
| Example 7 | 0.5-62 |

In the presence of liposomes having a particle diameter in the range of 1-10 μm of 53%, it was confirmed that the cell growth-promoting effect was obtained in a wide concentration range (Example 5).

Also, it was confirmed that the cell growth-promoting effect was obtained in a wide concentration range when the concentration of the sugar was 0.5% by weight or 50% by weight (Examples 6 and 7)

### Example 8 to 12

PQQ disodium (0.3 g), COATSOME NC-21 (hydrogenated soybean phospholipid) (3.0 g) and sugar at weights shown in Table 6 were mixed with water so that the total amount was adjusted to 100 g. In this case, the pH of the solution was 3.5, and the temperature was 60° C. The treatment of the solution obtained by a NISSEI AM-3 homogenizer was conducted under the following conditions to prepare a liposome composition in Examples 8 to 12.

### Example 8

treatment at a speed of 7000 rpm for 1 hour while warming at 80° C. or higher

### Example 9

treatment at a speed of 7000 rpm for 30 minutes while warming at 40° C. or higher

### Example 10

treatment at a speed of 7000 rpm for 30 minutes while warming at 60° C. or higher, and then the temperature was reduced to room temperature followed by treatment at a speed of 7000 rpm for 1 hour

### Examples 11 and 12

treatment at a speed of 7000 rpm for 30 minutes while warming at 60° C. or higher

The results of measurement of the particle diameter of the liposomes prepared in Examples 8 to 12 are shown in the following Table 6.

TABLE 6
Measurement result of the particle diameter

| | | Particle | | Proportion |
|---|---|---|---|---|
| | | diameter- | Size- | of particles |
| | | Normal | Normal | between |
| | | distribution- | distribution- | 1-10 μm |
| | Sugar | 50% (μm) | σg | (%) |
| Example 8 | Sorbitol | 2.989 | 1.541 | 99.36 |
| | 10% | | | |
| Example 9 | Sorbitol | 0.854 | 1.619 | 49.55 |
| | 10% | | | |
| Example 10 | Sorbitol | 2.547 | 1.886 | 94.21 |
| | 10% | | | |
| Example 11 | Sucrose | 1.969 | 1.573 | 95.57 |
| | 10% | | | |
| Example 12 | Glucose | 3.345 | 1.489 | 100 |
| | 10% | | | |

The results of the proliferation test of Examples 8 to 12 are shown in the following Table 7.

TABLE 7

| | | Growth-promoting |
|---|---|---|
| | | concentration |
| | Sugar | (μm) |
| Example 8 | Sorbitol 10% | 0.5-500 |
| Example 9 | Sorbitol 10% | 62-500 |
| Example 10 | Sorbitol 10% | 16-500 |
| Example 11 | Sucrose 10% | 1-250 |
| Example 12 | Glucose 10% | 4-250 |

In the presence of the liposomes having a particle diameter in the range of 1-10 μm of 49.55%, it was confirmed that the growth-promoting effect was observed in a wide concentration range (Example 9).

Regarding the sugar used, it was confirmed that the growth-promoting effect was observed by the addition of not only a sugar alcohol but also a monosaccharide and a disaccharide in a wide concentration range (Examples 11 and 12).

### INDUSTRIAL APPLICABILITY

The present invention can be beneficially used in the field of cosmetic products, food products, medicine, pesticides, etc.

1. A liposome composition, comprising:
liposomes, each of which comprises pyrroloquinoline quinone of formula (I): 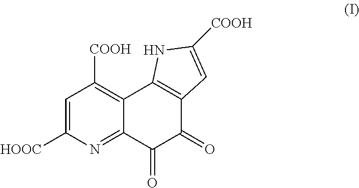 [SMILES: O=C(O)c1cc(C(=O)O)c2c(n1)C(=O)C(=O)c1cc(C(=O)O)[nH]c1-2]
or a salt thereof, and
sugar,
wherein 45% or more of the liposomes have a particle diameter of from 1 to 10 μm.

2. The liposome composition according to claim 1, wherein the liposomes have a volume mean particle diameter of from 0.5 to 20 μm.

3. The liposome composition according to claim 1, wherein the sugar is selected from the group consisting of a monosaccharide, a disaccharide, an oligosaccharide, a polysaccharide, and a sugar alcohol.

4. The liposome composition according to claim 1, wherein the sugar is sorbitol or xylitol.

5. A food product comprising the liposome composition according to claim 1.

6. A pharmaceutical product comprising the liposome composition according to claim 1.

7. A culture medium comprising the liposome composition according to claim 1.

8. A method of producing a liposome composition, the method comprising:
preparing a solution comprising pyrroloquinoline quinone of formula (I): 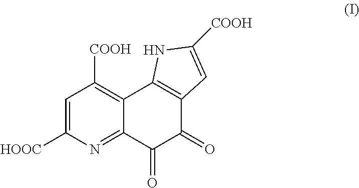 [SMILES: O=C(O)c1cc(C(=O)O)c2c(n1)C(=O)C(=O)c1cc(C(=O)O)[nH]c1-2]
or a salt thereof, sugar, and a lipid component at a temperature of from 40 to 190° C., thereby obtaining the liposome composition.

9. The method according to claim 8, wherein the solution has pH of 8 or lower.

10. The method according to claim 8, wherein
a concentration of pyrroloquinoline quinone or a salt thereof in the solution is of from 0.0001 to 2% by weight, and
a concentration of the sugar is of from 0.5 to 50% by weight.

11. The method according to claim 8, wherein the solution comprises from 1 to 200 parts by weight of the sugar and from 0.1 to 30 parts by weight of the lipid component per part of pyrroloquinoline quinone or a salt thereof.

12. The method according to claim 8, further comprising:
homogenizing the solution.

13. A liposome composition prepared by the method according to claim 8.
